(19) United States
(12) Patent Application Publication
(10) Pub. No.: US 2010/0159532 A1
(43) Pub. Date: Jun. 24, 2010

(54) METHOD AND DEVICE FOR PERFORMING A NUCLEIC ACID PREPARATION AND/OR AMPLIFICATION

(75) Inventors: Metin COLPAN, Essen (DE); Ralf Himmelreich, Langenfeld (DE); Thomas Rothmann, Langenfeld (DE)

Correspondence Address: Baker Donelson Bearman, Caldwell & Berkowitz, PC, 555 Eleventh Street, NW, Sixth Floor, Washington, DC, 20004, US

(73) Assignee: QIAGEN GMBH, Hilden (DE)

(21) Appl. No.: 12/640,798

(22) Filed: Dec. 17, 2009

(30) Foreign Application Priority Data
Dec. 23, 2008 (EP) 08172670.5

(51) Int. Cl. C12P 19/34; C12M 1/40

(52) U.S. Cl. 435/91.5; 435/303.1

(57) ABSTRACT

The invention relates to a method and a device for performing a nucleic acid preparation and/or amplification.

For the implementation of the method, a flexible tube is provided, which is always sealed below and hence at one end during the implementation of the sample preparation and/or amplification. In this tube, a biological sample is prepared and/or amplified. The use of only one tube is very economical. The tube is a disposable item which after a preparation or amplification can with its contents be disposed of environmentally correctly. Since moreover the tube has only to be filled with sample substances and reagents at a given time and manipulated, the method can also be simply and economically performed automatically.

The invention relates to a method and a device for performing a nucleic acid preparation and/or amplification.

From the state of the art it is known that for the preparation of a biological sample, firstly the contents of a biological sample are made accessible (known by the term “lysis” or “digestion”), components of the liberated contents of the biological sample are selectively bound onto a solid support or carrier material (known by the term “binding”), undesired components are removed from the solid support or carrier material (known by the term “washing”) and the desired components, namely nucleic acids, are then released from the solid support or carrier material (known by the term “elution”). Finally, at least one desired component, i.e. a defined part of a DNA or RNA strand, is multiplied, i.e. amplified, for example by means of a polymerase chain reaction (PCR).

An automated method for the preparation of a biological sample which includes the steps lysis, binding, washing, elution and PCR can be found in the European patent application with the official file number EP 08151152.9: a biological sample is placed in a container which has a filter for example consisting of a silica gel. Below the filter, an inlet and outlet respectively for liquid are provided. Firstly, in particular a biological sample is digested in this container with a lysis buffer. After the digestion of the sample, the lysis buffer is drawn down through the filter. Thereby, the nucleic acid liberated by digestion is at the same time bound onto the filter. The binding of the nucleic acid onto the filter is strengthened by subsequent addition of ethanol to the vessel. Washing is then performed using washing buffers, in order to remove fats, lipids and proteins. After the washing, the bound nucleic acid is detached from the filter using elution buffer. The elution buffer together with the nucleic acids now present therein is fed into a PCR chamber in order to perform the desired polymerase chain reaction therein.

It can also be seen from the European patent application with the official file number EP08151152.9 that the product of the PCR is separated by electrophoresis using an agarose gel and the separated nucleic acid fragments are stained in a water-bath containing ethidium bromide. By excitation with UV light, the stained nucleic acid fragments become visible and are thus detected. The binding takes place on the basis of “chaotropic” chemistry. Binding is then performed at high salt (i.e. a solution with high salt concentration). An ethanol elution is performed at low salt, i.e. a solution with low salt concentration. If on the other hand an anion exchanger is used, then the binding takes place at low salt and elution at high salt.

From the publication WO2008/006500, a device is known with which the nucleic acid can be prepared and amplified automatically. The device comprises a plurality of workstations for preparing and amplifying a liquid biological sample such as plasma and blood pipetted into a disposable container. The disposable container comprises various chambers, through which the liquid sample is transported by means of compressed air. Some of the reagents required for the preparation and amplification are pumped into the disposable container from the device, and some reagents or solid substances are present in the disposable container from the start. Reagents no longer required can be transported back into the device from the disposable container.

Admittedly the production of the disposable containers known here-from is already relatively inexpensive compared to other known disposable containers which are provided for performing a nucleic acid preparation and/or amplification, since on the basis of a device with the different workstations a relatively simply constructed container suffices. However, very expensive injection moulds must still be produced for the production of the disposable container. The device with the different workstations is also relatively expensive. Thus the state of the art does not make do with pipetting alone. If waste reagents are removed from the disposable container, then the risk of contamination is associated therewith. There is also a threat of contamination since the substances in the disposable container are not always closed off from the environment.

On the internet page http://www.devicelink.com/ivdt/archive/04/07/011.html and in the publication US 2004/0161788 A1, sample preparation and sample amplification which are carried out in a tubular container are described. The tubular container is subdivided into individual segments. The individual segments are firstly watertightly separated from one another by seals. The seals can be broken open such that two segments are connected with each other. Firstly, segments are filled with reagents. The sample preparation and sample amplification are performed by pressing segments together so that inter alia seals are broken open and segments appropriately connected with one another. In addition, using plungers, subdivision into segments can be effected in order to perform the desired process.

Since the tubular container must be prefilled and subdivided into segments, it is relatively expensive to produce. Disadvantageously, seals have to be broken open, which can result in malfunctions if they are incorrectly broken open.

Reagents no longer required are transported upwards in the tubular container by appropriate compression of the container and thus pass into a closure cap of the container, with which the container is sealed. A disadvantage here is that contamination can arise because of leakages between cap and container. Also a problem in this state of the art is that inter alia the fluid transport in the container is controlled by pressure. Thus for example reagents that are no longer required are conveyed into the closure cap by means of pressure. This increases the risk of the surroundings being contaminated owing to leakages. In the aforesaid state of the art with the tubular container no pressure equalization with the surroundings takes place, which renders the system is liable to malfunction.

The objective of the invention is to configure the method, including the device, more simply and in an improved manner.

To meet the objective, a flexible tube is provided which is always closed below and thus on one side during the implementation of the sample preparation and/or amplification. In this tube, a biological sample is prepared and/or amplified. The use of only one tube is very economical. The tube is a disposable item, which including its content can be disposed of environmentally compatibly after a preparation or amplification. Since moreover the tube has only to be filled with sample, substances and reagents and operated at a given time, the method can also be simply and economically performed automatically.

In contrast to the state of the art known from the publication US 2004/0161788 A1, the tube is not subdivided into segments which are initially sealed off from one another. Also, it is not necessary initially to prefill the tube with reagents or substances.

In one embodiment of the invention, in a first step a sample is introduced, for example pipetted, into the tube. Next, the tube is tightly compressed above the sample introduced and thus the sample present in the tube is closed off from the environment. Following this, in the subsequent steps the tube is operated in such a manner that the content already introduced into the tube is always closed off from the environment. If reagents or other substances required for performing the method are added, then this takes place by a lock principle, which ensures that the remaining content of the tube is at all times closed off from the environment so that no contamination can arise.

The method can however for example also be started by initially introducing a first reagent, in particular a lysis buffer, into the tube and the biological sample only after that.

In order to effect the inward transfer of a substance of a reagent, the substance or the reagent is introduced from above into the tube, which is tightly compressed above the other contents. After the introduction, the tube is tightly compressed above the substance introduced or above the reagent introduced. Next, the compressed part of the tube located thereunder is released from load and thus opened, so that the substance or the reagent is transported downward by gravity further into the tube into the desired region. Since the transport is effected by gravity, no overpressure has to be created in the tube.

For the implementation of the method, it suffices to introduce reagents or other substances. Robot technology utilized, such as an automatic pipetting device, has only to introduce reagents or substances appropriately, i.e. preferably pipette them, into the tube. For this, a tube is for example immovably mounted. By means of robot technology known per se, the given desired reagents or substances are withdrawn from supply vessels and pipetted into the tube at a given time.

The method for performing a sample preparation and amplification requires only that tube regions can be opened and closed by compression and release, and separation is effected at a given time by means of a magnet, and that heating and cooling can be effected for performing an amplification, as can be learned from the following explanations. Reagents and substances do not have perforce to be introduced by the lock principle in order to obtain good results. However, the lock principle is advantageous in order to avoid contamination.

The tube preferably consists of a suitable inert material such as silicone. In addition, the internal wall of the tube is preferably treated so that beads can slip along the internal wall more easily. For this purpose, the tube inner wall is for example silanized.

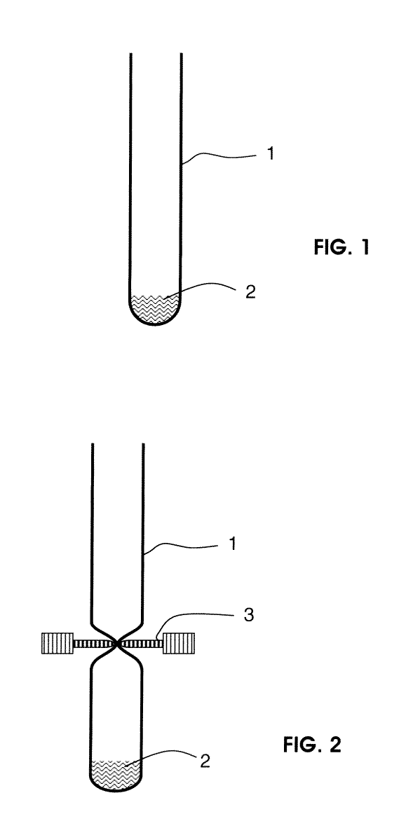

FIG. 1 shows a cross-section of a tube 1 which is closed below. A liquid biological sample 2 was introduced into this tube 1. Next, the tube is compressed above the sample by pressure devices 3, as illustrated in FIG. 2. The sample 2 is now closed off from the environment and cannot contaminate this.

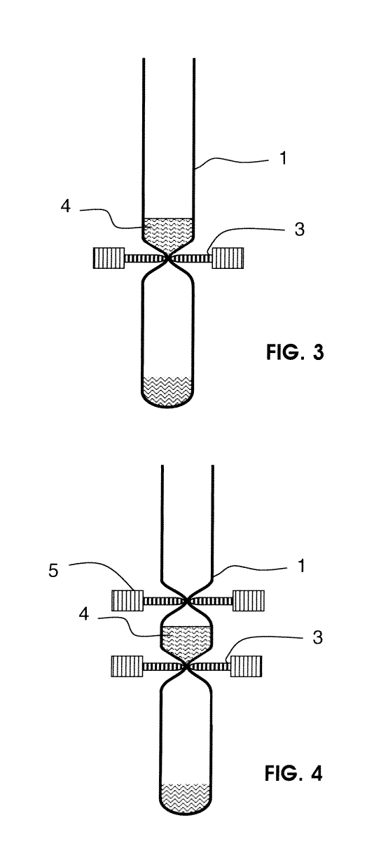

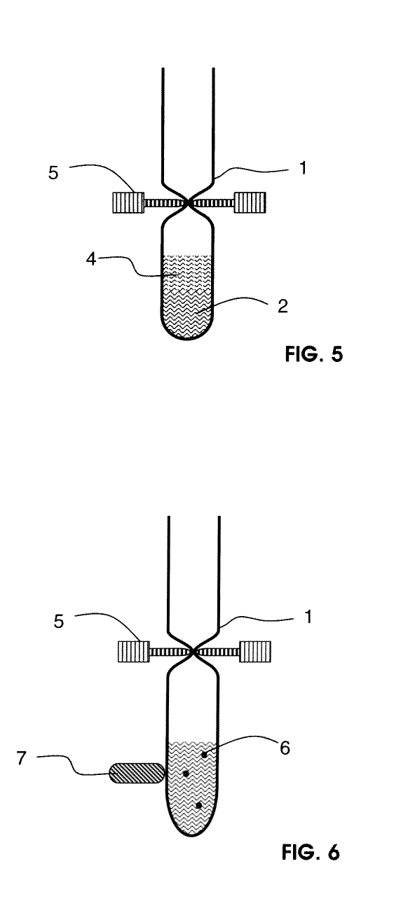

After the compression, a lysis buffer 4 is introduced into the tube, as shown in FIG. 3. Following the introduction of the lysis buffer, the tube is sealed above the buffer by pressure devices 5, as illustrated in FIG. 4. FIGS. 3 to 5 illustrate the lock principle, i.e. how a reagent or a substance is introduced into a desired region of the tube 1, without the other content of the tube being able to pass into the environment.

In order to promote the lysis, the lower region of the tube in one embodiment of the invention is heated, preferably to temperatures between 37° C. and 70° C.

In a further embodiment of the invention, the region of the tube in which the sample 2 and the lysis buffer 4 are situated is alternately partly compressed and again released for example by the pressure devices 3, in order to mix the lysis buffer with the sample. Here it is not necessary, and also not desirable, to compress the tube tightly, since the intention is not to seal off, but rather only to create turbulence in the two liquids for the purpose of mixing.

In one embodiment of the invention, the lysis buffer used has a salt content of less than 50 millimolar overall. It has been found that the observance of this limit can have a beneficial effect on the PCR sensitivity. This embodiment is advantageous mainly when an elution is not performed in the context of a sample preparation.

The pH value of the lysis buffer used is preferably adjusted to values between pH 7.5 and 9.5. This increases the PCR sensitivity if amplification is performed on beads. In order to stabilize the pH value in said manner, commercially available buffers Tris, Hepes and/or Mops are preferably used, in particular at a concentration of 10 to 20 millimolar, in order to achieve good PCR sensitivities. The aforesaid names are abbreviations which are familiar to the person skilled in the art.

As detergents for performing of the lysis in one embodiment of the invention, nonionic ones are used. Suitable nonionic detergents are sold commercially under the trade names Tween®, Nonidet P-40, Triton X-100 or Brij®. This selection also makes it possible to achieve good PCR sensitivities. The concentration of nonionic detergents is preferably 0.1 to 0.4 vol. %.

Optionally, polyvinylpyrrolidone (abbreviated as: PVP), in particular from 0.05 up to 0.1 vol. %, is added to the lysis buffer. Inhibitors are bound by PVP, hence still further improved PCR sensitivity is obtained.

Following the lysis, other substances are added, in particular firstly magnetic particles, which are called magnetic beads, to which the nucleic acid liberated by the lysis binds. In addition, substances can be added which promote this binding of the nucleic acid to the beads. This is always effected according to the lock principle. Thus firstly the relevant reagents or substances are pipetted into the tube which is tightly compressed above its content. Next the tube is tightly compressed above the last added substance or above the newly added reagent. Next, the closed tube region below the last added substance or above the newly added reagent is released from load in order to introduce the desired substances and reagents into the region located thereunder.

As beads to which the nucleic acid binds, magnetic silica beads, which are for example known from the publication EP 1 337 541 B1, are preferably used. However, alternatively, apart from the preferred magnetic silica beads, magnetic particles with ion exchanger surfaces, in particular with an anion exchanger surface can also be used to bind the released nucleic acid onto magnetic particles. Admittedly according to the state of the art the binding using ion exchange is attended by the disadvantage that the elution necessitates the use of salts at high concentration, which renders desalting necessary following the elution. However, if the elution is inapplicable, then subsequent desalting is also inapplicable.

Magnetic beads can be introduced into the tube immediately, before sample or lysis buffer is added. This embodiment of the invention helps to make it possible to implement the method rapidly.

FIG. 6 shows the situation wherein nucleic acids have been bound onto magnetic particles 7 and are situated in the lower region of the tube within the liquid present there. A magnet 7 is now brought up to the tube 1, in order to be able to collect the magnetic particles 6 and transport them upwards. Hence the tube is preferably pressed in by the magnet in order to shorten the distance between the magnet 7 and the particles 6 and thus to ensure that the magnetic particles are collected completely. For the same reason, the magnet 7 is appropriately moved up and down adjacent to the region with the liquid present therein.

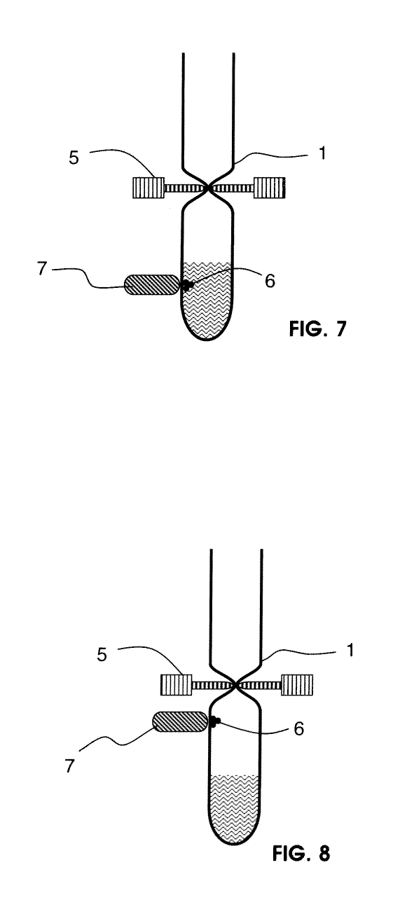

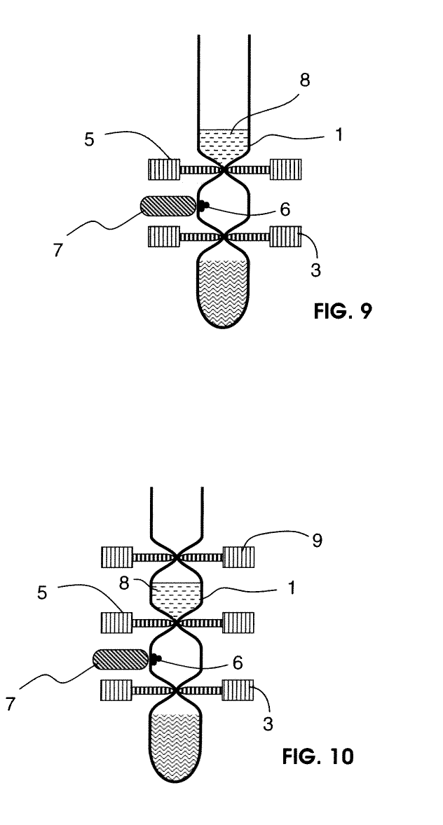

Finally, if the magnetic particles have been collected as sketched in FIG. 7, then the magnet is moved upward adjacent to the tube, as illustrated in FIG. 8. Below the magnetic particles thus transported out of the liquid, the tube is once again closed by compression. The reagents now no longer required remain in the tube closed off from the outside and cannot harmfully escape into the surroundings. Hence the lower closed off region serves as a chamber for accommodating the waste products from the method. Above this there is a closed off chamber or a closed off region in which the magnetic particles with the nucleic acids bound onto them are situated, as shown in FIG. 9.

Following this, washing buffers 8 are introduced according to the lock principle into the chamber with the magnetic particles present in it. Firstly a washing buffer is introduced, as shown in FIG. 9. The washing buffer is then situated above, and watertightly separate from the chamber with the magnetic particles 6 present in it. After the introduction of the washing buffer 8, the tube is sealed by means of pressure devices 9 above the washing buffer 8 introduced, as shown in FIG. 10. The pressure devices 5 situated under this are released, so that the tube relaxes, and the washing buffer 8 passes under gravity into the region with the magnetic particles 6 present in it. In one embodiment of the invention, this region of the tube is also compressed and again released from load by means of the pressure devices 5, in order to promote thorough mixing of the buffers with the magnetic particles. Advantageously, during the washing the magnet 7 is moved away from the tube, so that the particles can easily become distributed in the washing buffer. After the washing, the magnet is again brought up, in order to collect the magnetic particles again. In order to accelerate the collection, the tube is compressed in the relevant region for example by means of the magnet and/or the magnet is moved upward and downward along the relevant region. The tube can also be rotated about its longitudinal axis together with the relevant pressure devices, in order to ensure complete collection of the magnetic particles 6. Conversely, the magnet can be moved around the tube in order better to achieve this.

If the magnetic particles have been collected by the magnet and if these are held on a side wall by the external magnet 7, then the pressure device 3 can be opened, in order to allow the now no longer required washing buffer to flow down into the lower region in which the wastes are collected and kept. Next, the relevant tube region is again sealed by means of the pressure device 3.

The sample components bound onto the magnetic particles can now be immediately amplified, preferably by a two-stage amplification method such as for example a nested PCR, if no elution was performed beforehand. It has been found that in the case of a two-stage amplification it is not absolutely necessary for an elution to be performed after the washing in order to obtain good results.

In order to perform the amplification, the magnetic particles can be taken out of the tube into a separate chamber for performing an amplification.

In one embodiment, an elution is firstly performed after the washing. For this, the appropriate elution buffers are added by means of the lock principle to the magnetic particles with the nucleic acids bound on them. Advantageously, for performing the elution, the magnet 7 is removed from the tube so that the magnetic particles can become distributed in the elution buffer without difficulty. Through the elution, the nucleic acid is detached from the magnetic beads. If silica beads were used for the binding, then a low salt is preferably used as the elution buffer. The elution can again be accelerated by compressing and releasing the relevant region of the tube, in order to create turbulence in the elution buffer. After performing the elution, it is advisable to separate the magnetic beads. This is for example effected by means of the magnet 7 in the manner already described.

Should only a sample preparation be performed, then this is completed at the latest after completion of the elution. The elution buffer with the nucleic acid present in it can now be taken out of the tube and further used in the desired manner. During this, in particular the lower tube region with the wastes present in it is kept closed. If the elution buffer has been removed, then the tube can now for example be closed in an upper region with a clamp. Now pressure devices can be released and the tube including content and clamp suitably disposed of, without there being a risk existing of contaminating the environment.

It is however also possible to perform an amplification, for example a PCR, in the tube after completion of the sample preparation. In this case, the tube must at first be kept closed.

The practical example so far explained on the basis of FIGS. 1 to 10 requires a relatively long tube and an arrangement of pressure devices along this tube. In order to be able to arrange the pressure devices, the magnet and any heating devices on a relatively small region, the embodiment described below is preferred.

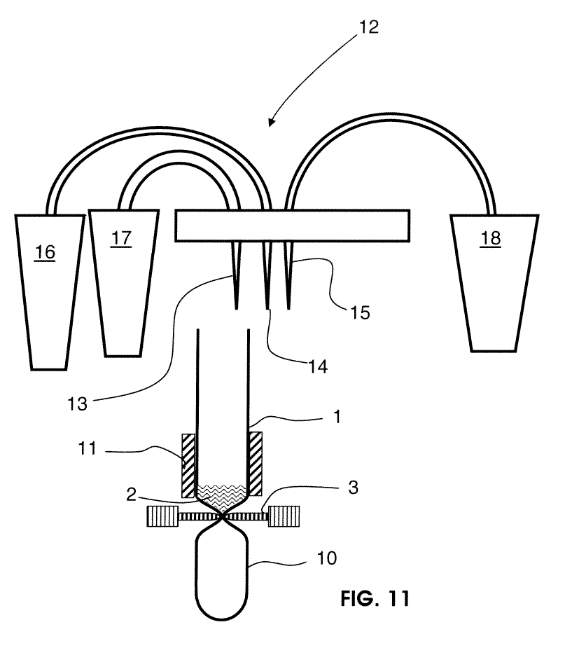

Firstly a lower region of the tube is sealed by compression of the tube. Next, the sample is introduced into the tube. The sample now does not reach the bottom of the tube, but only the bottom of the region which is situated above the compressed part of the tube 1, as is illustrated by FIG. 11. Following this, the desired steps for performing a preparation and/or amplification are performed as already described. In contrast to the previous embodiment, however, a separation of lysis buffer and magnetic beads through the magnetic beads being transported upwards at a given time by means of a magnet does not take place. Instead of this, the beads are only held by a magnet 7 on a side wall of the tube 1 above a lower region 10. The lower region 10 is firstly separated from tube regions lying under it by a part of tube tightly compressed by pressure devices 3. Next, the pressure devices 3 are opened and the lysis buffer which is no longer required flows out downwards. After the lysis buffer has thus been separated from the magnetic beads, the lower region is again closed by the pressure devices 3 and the process continued in the manner described.

In FIG. 11, cooling and heating devices adjacent to an upper region of the tube, which are capable of cooling and heating, are shown. By means of the cooling and heating devices 11, the adjacent region of the tube can be compressed and again released from load, in order thus for example also to accelerate a lysis or an amplification mechanically. The cooling and heating devices 11 are sized and configured such that a large heat transfer area is available.

The tube is installed within a device which includes an automatic pipetting device 12. The automatic pipetting device has a plurality of movable injection needles 13, 14 and 15, via which required liquids are passed into the tube, in particular automatically. Each injection needle is connected to a supply vessel 16, 17 and 18, in which the reagents and substances required in order to be able to perform the sample preparation and/or amplification are located.

In one embodiment of the invention, an oil film can be provided in order better to ensure that no contamination arises in the event of incorrect operation or other malfunctions. The oil film floating on buffers then acts as an additional seal or closure so as not to allow substances and reagents situated in the container to reach the exterior.

If an amplification is to be performed in the tube following the sample preparation, then a PCR buffer is introduced by the lock principle into the tube and thus added to the elution buffer with the sample present in it.

If the magnetic beads would impede an amplification, these can be removed upwards from the tube by the lock principle by means of a magnet. Preferably however, in order to avoid all contamination risk, the magnetic beads in such cases are collected at the bottom of the relevant region and the tube then tightly compressed immediately above them. Next, the compressed region of the tube below the magnetic beads is released and thus opened, in order then to allow the magnetic beads to fall into the waste region for example by removal of the magnet. Admittedly in this embodiment a small fraction of already prepared sample is lost. In return, contamination is better prevented compared to the case where the magnetic beads are removed from the tube.

As a rule, however, the magnetic beads remain in the region of the tube in which the PCR is performed, since the presence of magnetic beads does not in principle impede the amplification.

During the amplification the tube is advantageously compressed by heating and cooling devices 11 such that the heat transfer area is thereby increased. As heating and cooling devices, electrothermic transducers (Peltier elements) are preferably used, in order to be able both to heat and cool without difficulty. This embodiment is advantageous since in a PCR heat must be rapidly supplied and removed in order to obtain good results.

At the same time, during the amplification the relevant tube region can be compressed and expanded several times, in order to create turbulence and hence to promote the heat exchange. Preferably in this embodiment the cooling and heating devices are again used for appropriately deforming the tube region.

A real time PCR can also be performed in the tube. For this purpose, the tube has a window in the relevant region, or the tube is completely transparent. During the amplification, changes in fluorescence are determined by means of an optical system, in order to perform a real time PCR in a known manner.

An amplification of a biological sample can also advantageously be performed in a flexible, differently shaped container, which is compressed one or several times before or during the amplification, preferably by means of heating and/or cooling devices, in order to accelerate the amplification. Thus the container does not necessarily have to be a tube sealed at one end in order to promote the amplification through the single or multiple compression alternating with release for the aforesaid reasons.

Experimentally, the following experiments inter alia were performed.

A silanized or silicone tube of 1 cm internal diameter was used. As a result of the silanization, magnetic beads slip especially easily along the internal wall of the tube, which facilitates the transport of the magnetic beads by means of an external magnet. For the silanization, a 2-5% w/v dimethyldichlorosilane solution in a suitable organic solvent (e.g. cyclotetrasiloxane) was used. The person skilled in the art will find silanization solutions under the CAS number: “17-1332-01”. For the silanization, a ca. 12 cm long tube section was closed at one end using a tube clamp, and filled with the solution. The solution can be poured out again immediately. After release of the tube clamp, the tube was dried for one hour in a fume hood.

### Experiment A/Nucleic Isolation Process A:

In this preparation, the lower tube end was permanently closed with a tube clamp. In addition, a further tube clamp was required in order to create a new preparation zone by clamping after each liquid exchange and the “pushing out” of the magnetic silica beads from the liquid. This procedure is repeated several times. The “pushing out” of the magnetic silica beads from the liquid was effected using a strong permanent magnet.

Practical Implementation:

200 μl of blood and 200 μl of buffer AL and 20 μl of proteinase K were pipetted into the tube. Next, the tube was hung for 15 mins in a water-bath warmed to 56° C. After the incubation, 200 μl of isopropanol and 15 μl of MagAttract Suspension G were added. By multiple (10-20×) compression and relaxation of the tube, the contents were mixed. For a period of 3 mins, the mixing was repeated every 20 secs. Now a strong permanent magnet was placed against the liquid from the outside and the magnetic beads were magnetically sedimented for 20 secs. The magnet was slowly shifted upwards by 2-3 cm, until it was outside the liquid. Now the region between the liquid meniscus and the position of the magnet was tightly compressed with a second tube clamp. In this way a new reaction zone was now created. After removal of the permanent magnet, 1 ml of washing buffer Buffer AW1 was introduced into the tube from above. By multiple (10-20×) compression and relaxation of the tube, the magnetic particles were again resuspended. Now the permanent magnet was again placed against the liquid from the outside and the magnetic beads sedimented magnetically for 20 secs. Once again, the magnet was slowly shifted upwards by 2-3 cm, until the magnetic particles were outside the liquid. By means of a third tube clamp, which was placed between the liquid meniscus and the position of the magnet, a reaction zone situated just above was created. The washing procedure was repeated one further time with the washing buffer Buffer AW2. This time, the clamping off of a new reaction zone could be effected by releasing and applying the “second” tube clamp. For the elution, the magnetic particles were now located in a new reaction zone and liquid residues of the washing buffer were as far as possible removed. Before the elution, the magnetic beads were dried for 5 mins with a gentle air current in order to remove residues of ethanol-containing washing buffer. Now elution with 200 μl of RNase-free water was performed by addition from above. For this the tube was continuously compressed and again released for a period of 1 min. Thereby the magnetic particle sediment was resuspended and the bound nucleic acids were eluted. As a final step, a strong permanent magnet was applied above the middle tube clamp from outside and the magnetic silica beads were magnetically sedimented for 20 secs. By a back pressure (i.e. from the opposite side) by means of a further magnet or suitable object, the magnetic sediment was wedged in and the liquid (eluate) transferred into a new reaction zone or withdrawal zone lying above this. This eluate freed from magnetic particles was now withdrawn and analyzed. The trade names used in this paragraph are marks or trade names of the firm QIAGEN GmbH of Hilden, Germany. The products are marketed commercially under these names by QIAGEN GmbH.

### Experiment B/Nucleic Isolation Process B)

In this preparation, the lower tube end was permanently closed with a tube clamp. A further tube clamp was placed in the middle of the tube. The preparation zone was located above the upper clamp. For liquid exchange, the magnetic silica beads were held fixed by means of a strong permanent magnet. By release of the middle tube clamp, the liquid simply ran into the lower region.

### Practical Implementation:

The upper tube part was filled with 200 μl of blood and 200 μl of Buffer AL and 20 μl of proteinase K and hung for 15 mins in a water-bath warmed to 56° C. After the incubation, 200 μl of isopropanol and 15 μl of MagAttract Suspension G were added. By multiple (10-20×) compression and relaxation of the tube, the contents were mixed. For a period of 3 mins, the mixing was repeated every 20 secs. Now a strong permanent magnet was applied from outside above the middle tube clamp and the magnetic silica beads were magnetically sedimented for 20 secs. While the magnet held the silica beads in position, the middle tube clamp was released and the liquid ran downwards into the lower region. The middle tube band clamp was again closed. Now the permanent magnet was removed and 1 ml of washing buffer Buffer AW1 introduced into the tube from above and the magnetic particles were again resuspended by multiple compression (ca. 10-20 times) and relaxation of the tube. The magnetic separation and the removal of the washing buffer proceeded as described above. The washing procedure was repeated a further time with the washing buffer Buffer AW2. After the washing buffer had been passed into the lower region and the middle tube clamp had been shut, the so-called “water rinse” was performed. For this, the permanent magnet remains in position and swirling the magnetic particle sediment is avoided. In the water rinse, 1 ml, of water was cautiously introduced into the tube from above. After 5 secs the middle tube clamp was released and the water ran downwards into the lower region. During this procedure, the magnet held the silica beads in position the whole time. The middle tube clamp was now closed again and the elution with 200 μl of RNase-free water was performed by addition from above. For this the tube was continuously compressed and again released for a period of 1 min. Thereby the magnetic particle sediment was resuspended and the bound nucleic acids were eluted. As a final step, a strong permanent magnet was applied above the middle tube clamp from outside and the magnetic silica beads were magnetically sedimented for 20 secs. The supernatant was now pipetted off from above and contained the purified nucleic acids, as was found by a subsequent analysis.

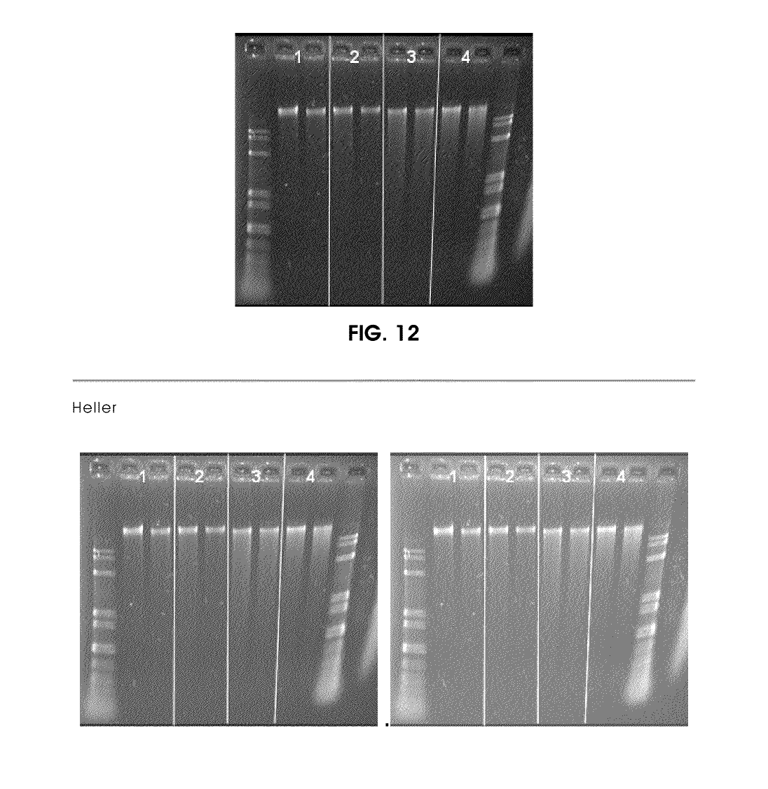

FIG. 12 illustrates the result of an electrophoretic analysis (0.8% agarose gel, 8 μl eluate applied per slot)

1: Tube preparations with “lower waste” strategy (Experiment B)
2: Tube preparations with “moving magnets” strategy (Experiment A)
3: Reference preparation performed by means of the QIAamp Blood DNA Mini Kits (from QIAGEN GmbH of Hilden)
4: Reference preparation performed by means of QIAGEN BioSprint 15

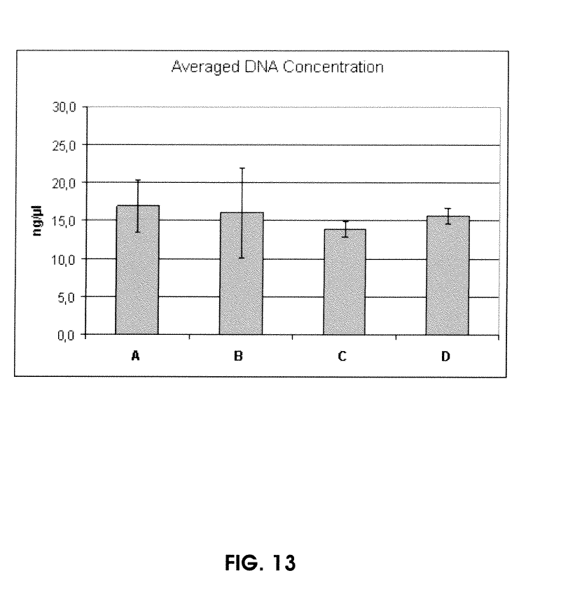

FIG. 13 shows the result of the DNA quantification by UV spectrometry

A: Tube preparations with “lower waste” strategy (Nucleic Isolation Process B)
B: Tube preparations with “moving magnets” strategy (Nucleic Isolation Process A)
C: Reference preparation performed by means of the QIAamp Blood DNA Mini Kits (QIAGEN)
D: Reference preparation performed by means of QIAGEN BioSprint 15

### CONCLUSIONS

FIGS. 12 and 13 show that the sample preparation in a tube yields comparable results to standard reference methods. As a result of the new materials and procedures, new strategies for nucleic acid preparation can be implemented.

1. A method for performing a preparation and/or amplification of a biological sample, wherein a biological sample is prepared and/or amplified in a tube sealed at one end.

2. The method as claimed in claim 1, wherein said biological sample is introduced into the tube, following which the tube above the sample is tightly compressed, next a reagent or a substance is introduced into the tube, the tube above the reagent introduced or the substance introduced is tightly compressed, next the tight connection below the reagent introduced or the substance introduced created by compression is opened.

3. The method as claimed in claim 2, wherein a lysis buffer, a washing buffer, an elution buffer, magnetic particles for the binding of nucleic acid and/or a buffer for the amplification of nucleic acid is used as said reagent and/or said substance.

4. The method as claimed in claim 1, wherein nucleic acid of said biological sample bound onto magnetic particles is separated from a reagent or a substance by means of a magnet, whereby the magnetic particles are collected and held by the magnet and following which the magnetic particles are drawn out of either the reagent or the substance by means of the magnet or the reagent or the substance is removed by opening of a tightly compressed region of the tube, which is situated below the held magnetic particles.

5. The method as claimed in claim 2, wherein reagent and/or substance no longer required is collected at a bottom portion of the tube.

6. The method as claimed in claim 1, wherein during digestion, washing, elution or amplification of said sample the tube is compressed and released from load once or several times by at least one pressure device, by a heating and/or cooling device and/or by a magnet.

7. The method as claimed in claim 1, wherein the tube does not comprise any segment or chamber subdivided by cohesively bonded seals and/or has not been prefilled with reagent or substance.

8. A method as claimed in claim 1, wherein an amplification of said biological sample is performed and the tube is compressed once or several times before or during the amplification.

9. A device suitable for implementation of a method as claimed in claim 1, comprising a tube, which is adapted to be sealed with at least one pressure device, in order to be able to compress the tube tightly at different heights.

10. The device as claimed in claim 9, comprising a magnet, which can from outside said device, be moved towards the tube and also away from the tube.

11. The device as claimed in claim 9, comprising an automatic pipetting device or a robotic pipetting system for introduction of liquid into the tube.

12. The device as claimed in claim 9, comprising at least one heating and/or cooling device with which the tube can be heated and/or cooled.

13. The device as claimed in claim 9, comprising a plurality of supply vessels capable of containing reagents or substances therein.

14. The device as claimed in claim 13, wherein a lysis buffer, a washing buffer, and elution buffer, magnetic particles for the binding of nucleic acids or a puffer for performing an amplification is present in at least one of said supply vessels.

15. A device of claim 9, comprising exactly three pressure devices.

16. A method of claim 6, comprising exactly three pressure devices.
